(19) United States
(12) Patent Application Publication
(10) Pub. No.: US 2007/0261700 A1
(43) Pub. Date: Nov. 15, 2007

(54) Vibrating condom

(76) Inventor: Tze-pin Chan, Taichung (TW)

Correspondence Address: SAM CHEN, 7F-1, 293, ROOSEVELT ROAD, SEC 3, TAIPEI, omitted

(21) Appl. No.: 11/736,011

(22) Filed: Apr. 17, 2007

(30) Foreign Application Priority Data
May 11, 2006 (TW) 095208032

(51) Int. Cl. A61F 6/04

(52) U.S. Cl. 128/844

(57) ABSTRACT

In one embodiment, a vibrating condom includes a flexible, thin inner sheath; an outer tube around the sheath, the tube being thicker and less flexible than the sheath; a vibrating unit projecting on an outer surface of the tube; a battery powered power source disposed in the vibrating unit; a DC motor disposed in the vibrating unit and electrically connected to the power source for vibrating the vibrating unit; a flexible, receptacle at forward ends of the sheath and the tube, the receptacle being thinner than the sheath; and an elastic band attached to rear ends of the sheath and the tube. Another embodiment of vibrating condom with an external battery powered power source is also disclosed.

### BACKGROUND OF THE INVENTION

1. Field of Invention

The invention relates to condoms and more particularly to a battery powered vibrating condom with improved characteristics including heightening mutual satisfaction and without causing premature ejaculation during sexual intercourse.

2. Description of Related Art

Condoms are well known protective and contraceptive devices. Moreover, a type of vibrating condom is commercially available recently. For example, U.S. Pat. No. 5,377,692 discloses a vibrating condom in which a vibrating region is attached to an anterior portion thereof. The device is designed to contact the clitoris and vibrate in two directions. The device is also designed to include a vibrating ring that contacts the vaginal walls.

In addition, U.S. Pat. No. 6,907,883 discloses a battery powered electric condom ring which can be put on the penis when in use or be used with a condom. Thus, continuing improvements in the exploitation of vibrating condom are constantly being sought.

### SUMMARY OF THE INVENTION

It is therefore one object of the invention to provide a battery powered vibrating condom for heightening mutual satisfaction without causing premature ejaculation during sexual intercourse.

The above and other objects, features and advantages of the invention will become apparent from the following detailed description taken with the accompanying drawings.

### BRIEF DESCRIPTION OF THE DRAWINGS

FIG. 1 is a longitudinal sectional view of a first preferred embodiment of vibrating condom according to the invention;

FIG. 2 is a cross sectional view of the condom;

FIG. 3 is a longitudinal sectional view of the vibrating region;

FIG. 4 is a cross sectional view of the vibrating region showing components thereof;

FIG. 5 is a longitudinal sectional view of a second preferred embodiment of vibrating condom according to the invention;

FIG. 6 is a longitudinal sectional view of the vibrating region attached to the power unit by a cord; and

FIG. 7 is a cross sectional view of the vibrating region attached to the power unit by the cord.

### DETAILED DESCRIPTION OF THE INVENTION

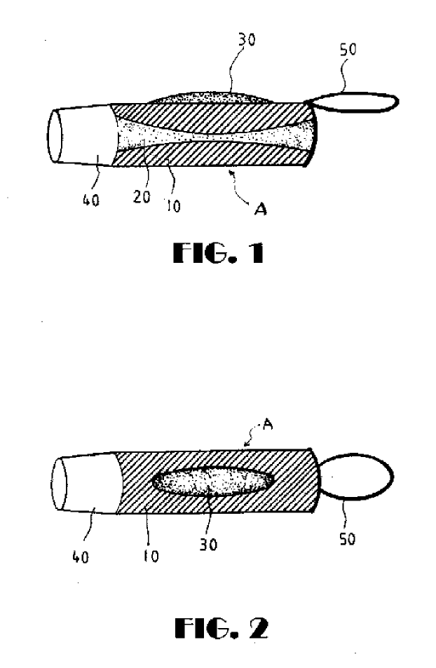

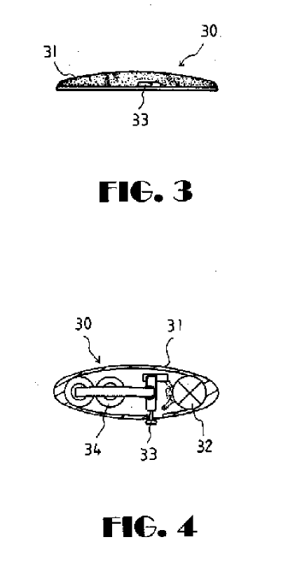

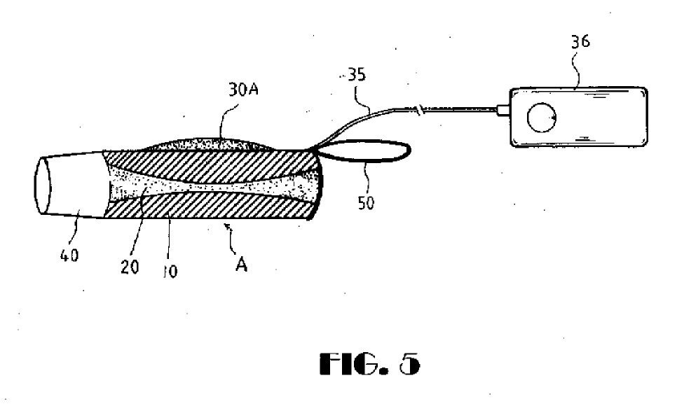

Referring to FIGS. 1 to 4, a vibrating condom A in accordance with a first preferred embodiment of the invention is shown. The condom A is shown in an un-rolled configuration. The condom A is formed integrally and comprises a thin, flexible inner sheath 20, a relatively thick outer tube 10 as a brace formed around the sheath 20, a raised vibrating region 30 of oval cross section formed on a top of the tube 10, a thinner, flexible anterior portion 40 formed at forward ends of both the sheath 20 and the tube 10, and an elastic band 50 attached to rear ends of both the sheath 20 and the tube 10.

The vibrating region 30 comprises a cover 31, one or more cells (two are shown) 34 such as coin-shaped lithium cells, a miniature DC (direct current) motor 33 electrically connected to the cells 34, and an on/off switch 33 provided on the cover 34 and being electrically interconnected the cells 34 and the motor 33.

In use, a male first put the vibrating condom A on the penis. Next, put the band 50 on the skin near the septum of scrotum. The band 50 then contracts to prevent the vibrating condom A from slipping out of the penis during sexual intercourse. Also, the tube 10 can help the vibrating condom A tighten the penis without slipping for an increased time period. Further, the anterior portion 40 is adapted to tightly fasten the glans penis without causing premature ejaculation. Next, turn on the switch 33 to activate the motor 33 which in turn vibrates the vibrating region 30 for stimulating the clitoris and the vaginal walls of a female partner. It is envisaged by the invention that the vibrating condom A can heighten mutual satisfaction during sexual intercourse.

Alternatively, a conventional condom can be put on the main portion of the vibrating condom A (i.e., except the band 50) during sexual intercourse if the male prefers.

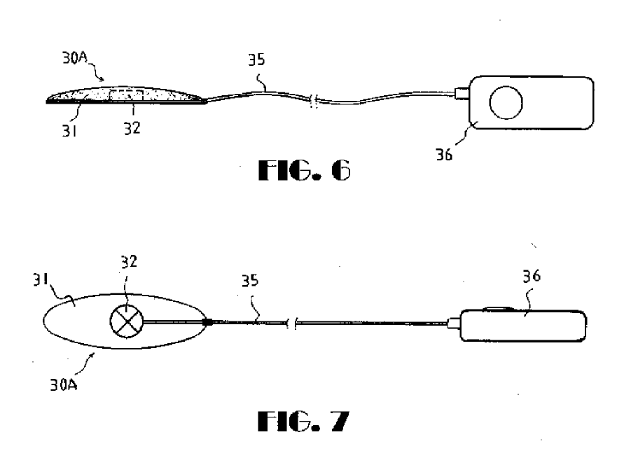

Referring to FIGS. 5 to 7, a vibrating condom A in accordance with a second preferred embodiment of the invention is shown. The characteristics of the second preferred embodiment are detailed below. The motor and the cells are eliminated such that a larger DC motor 32 can be mounted in the vibrating region 30A under the cover 31. Further, a large, powerful power unit (e.g., one having two connected cylindrical 1.5 volt “AA” cells) 36 is provided externally of the condom A. A cord 35 is electrically interconnected the motor 32 and the power unit 36. An on/off switch (not numbered) is provided on an outer surface of the power unit 36.

Advantages of the second preferred embodiment are detailed below. The vibration is even stronger. The operating time of the vibrating condom is longer. Cells of the power unit 36 can be replaced easily. Also, the cells can be rechargeable ones so as to be environment friendly.

While the invention herein disclosed has been described by means of specific embodiments, numerous modifications and variations could be made thereto by those skilled in the art without departing from the scope and spirit of the invention set forth in the claims.

What is claimed is:

1. A vibrating condom comprising:
a flexible, thin inner sheath;
an outer tube around the sheath, the tube being thicker and less flexible than the sheath;
vibrating means projecting on an outer surface of the tube;
a battery powered power source disposed in the vibrating means;
a DC (direct current) motor disposed in the vibrating means and electrically connected to the power source for vibrating the vibrating means;
a flexible, receptacle at forward ends of the sheath and the tube, the receptacle being thinner than the sheath; and
an elastic band attached to rear ends of the sheath and the tube.

2. A vibrating condom comprising:
a flexible, thin inner sheath;
an outer tube around the sheath, the tube being thicker and less flexible than the sheath;
vibrating means projecting on an outer surface of the tube;
a battery powered power source disposed externally of the vibrating means;
a DC (direct current) motor disposed in the vibrating means and electrically connected to the power source for vibrating the vibrating means;
a receptacle at forward ends of the sheath and the tube, the receptacle being thinner than the sheath; and
an elastic band attached to rear ends of the sheath and the tube.
